(19) United States
(12) Patent Application Publication
(10) Pub. No.: US 2014/0343344 A1
(43) Pub. Date: Nov. 20, 2014

(54) Radiation Therapy Guided Using Gamma Imaging

(71) Applicant: Cubresa Inc., Winnipeg (CA)

(72) Inventors: John Saunders, Winnipeg (CA); James Schellenberg, Winnipeg (CA)

(21) Appl. No.: 14/247,638

(22) Filed: Apr. 8, 2014

Related U.S. Application Data
(60) Provisional application No. 61/809,648, filed on Apr. 8, 2013.

(51) Int. Cl. A61N 5/10; A61B 6/03

(52) U.S. Cl. CPC A61N 5/107; A61N 5/1039; A61B 6/037
USPC 600/1

(57) ABSTRACT

Radiation therapy of a lesion within a patient is guided to take into account movement of the lesion caused by respiration and/or cardiac effects by using MRI or other imaging system suitable for locating the lesion to image the patient while on the treatment support and using a gamma imaging system responsive to a radiation source preferentially taken up by the lesion and registered with the MRI so as to monitor movement of the lesion in real time and thus guide the beam of the RT.

This application claims the benefit under 35 USC 119(e) of Provisional Application 61/809,648 filed Apr. 8, 2013.

This invention relates to a guided Radiation Therapy system using. GI (Gamma Imaging) in association with another imaging modality such as MRI (Magnetic Resonance Imaging) for location of a lesion for treatment and for controlling the radiation to the lesion.

Reference is made to the following applications of the present applicants, the disclosure of which is incorporated herein by reference
PCT/CA20131050113 filed Feb. 14, 2013
PCTCA2012/050423 filed Jun. 26, 2012
U.S. patent application Ser. No. 14/128,112 filed on Jun. 26, 2012
PCT/CA2011/050074 filed Feb. 10, 2011
Australian Patent Application 2011214864 filed Feb. 10, 2011
Canadian Patent Application 2,788,976 field Feb. 10, 2011
European Patent Application 11741794.9 filed Sep. 4, 2012
U.S. patent application Ser. No. 13/516,995 field Oct. 1, 2012

### BACKGROUND OF THE INVENTION

A radiotherapy device generally includes a linear electron beam accelerator which is mounted on a gantry and which can rotate about an axis which is generally parallel to the patient lying on the patient couch. During the radiation therapy, the patient is treated using either an electron beam or an X-Ray beam produced from the original electron beam. The electron or X-Ray beam is focused at a target volume in the patient by the combination of the use of a collimator and the rotation of the beam. The patient is placed on a couch which can be positioned such that the target lesion can be located in the plane of the electron beam as the gantry rotates in two directions.

The objective of the radiation therapy is to target the lesion with a high dose of radiation over time and to have minimal impact on all the surrounding normal tissue. The first task is to precisely locate the tumor in three dimensional space. The best technique for this is MRI since this technology provides high resolution in the imaging of soft tissue to provide high soft tissue contrast.

Even though MRI provides good location of the tumor at the time of the measurement, these images are normally recorded two to three days prior to the treatment and so may not be completely representative of tumor location on the day of treatment. This is because the movement of the patient over time can cause the anatomical location of the tumor to move. The oncologists therefore tend to increase the target volume to be certain that all of the tumor tissue receives the required dose of the radiation, even though this increase in the volume of the tissue exposed to radiation also necessarily targets healthy tissue with consequential damage to the healthy tissue. The expectation is that all cells in the targeted region will be killed and this includes both the lesion and the healthy tissue. This produces collateral damage and may have a significant impact of the quality of life of the patient.

An external beam radiotherapy device generally includes a linear electron beam or an X-Ray beam accelerator provider which is mounted on a gantry and which can rotate about an axis which is approximately parallel to the patient lying on the patient couch. The patient is treated using either an electron beam or an X-Ray beam produced from the original electron beam. The electron or X-Ray beam is focused at a target by the combination of the use of a collimator and the rotation of the beam. The patient is placed on a couch which can be positioned such that the target lesion can be located in the plane of the electron beam as the gantry rotates.

An additional challenge to effective radiation treatment is the effect of motion of the tumor in the body due to respiratory and cardiac motion. This results in tumor masses moving making the continuous accurate targeting for treatment difficult. Again therefore the oncologists generally increase the size of the target volume radiated to accommodate movement of the lesion during respiratory and cardiac movement.

A number of attempts have been made to compensate for the movement of the lesion during the irradiation.

U.S. Pat. No. 6,725,078 (Bucholz) assigned to St Louis University and issued Mar. 6, 2001 discloses a combined MRI and radiotherapy system which operate simultaneously but without interference so that the location of the lesion can be tracked during the radiotherapy.

U.S. Pat. No. 6,731,970 (Schlossbanner) assigned to BrainLab and issued May 4 2004 discloses a method for breath compensation in radiation therapy, where the movement of the target volume inside the patient is detected and tracked in real time during radiation by a movement detector. The tracking is done using implanted markers and ultrasound.

U.S. Pat. No. 6,898,456 (Erbel) assigned to BrainLab and issued May 24 2005 discloses method for determining the filling of a lung, wherein the movement of an anatomical structure which moves with breathing, or one or more points on the moving anatomical structure whose movement trajectory is highly correlated with lung filling, is detected with respect to the location of at least one anatomical structure which is not spatially affected by breathing, and wherein each distance between the structures is assigned a particular lung filling value. There is also disclosed a method for assisting in radiotherapy during movement of the radiation target due to breathing, wherein the association of lung filling values with the distance of the moving structure which is identifiable in an x-ray image and the structure which is not spatially affected by breathing is determined, the current position of the radiation target is detected on the basis of the lung filling value, and wherein radiation exposure is carried out, assisted by the known current position of the radiation target.

U.S. Pat. No. 7,265,356 (Pelizzari) assigned to University of Chicago and issued Sep. 4, 2007 discloses an image-guided radiotherapy apparatus and method in which a radiotherapy radiation source and a gamma ray photon imaging device are positioned with respect to a patient area so that a patient can be treated by a beam emitted from the radiotherapy apparatus and can have images taken by the gamma ray photon imaging device. Radiotherapy treatment and imaging can be performed substantially simultaneously and/or can be performed without moving the patient in some embodiments.

U.S. Pat. No. 7,356,112 (Brown) assigned to Elektra and issued Apr. 8, 2008 discloses that artifacts in the reconstructed volume data of cone beam CT systems can be removed by the application of respiration correlation techniques to the acquired projection images. To achieve this, the phase of the patients breathing is monitored while acquiring projection images continuously. On completion of the acquisition, projection images that have comparable breathing phases can be selected from the complete set, and these are used to reconstruct the volume data using similar techniques to those of conventional CT. This feature in the projection images can be used to control delivery of therapeutic radiation dependent on the patient's breathing cycle, to ensure that the tumor is in the correct position when the radiation is delivered.

The same company Elekta AB of Stockholm Sweden have developed a machine using CT guided radiation where CT is used to image the patient just prior to irradiation. They state that better margins can be set using Motion View sequential imaging.

There are previous proposals for using MRI magnets to monitor treatment using electron beams created by a linear accelerator. The problem with this is the non-compatibility of linear accelerators and MRI. This arises because the magnetic field generated by the magnet of course interferes with the operation of the linear accelerator to an extent which cannot be readily overcome. It has however been found that relatively low field MRI units can be used with gamma radiation produced from cobalt-60.

In U.S. Pat. No. 5,735,278 (Houllt et al) issued Apr. 7^{th} 1998, is disclosed a medical procedure where a magnet is movable relative to a patient and relative to other components of the system. The moving magnet system allows intra-operative MRI imaging to occur more easily in neurosurgery patients, and has additional applications for liver, breast, spine and cardiac surgery patients.

The company ViewRay has built a gamma knife inside a double donut magnet for real time imaging to localize and also monitor the effect of motion. They plan, at least in the first instance to use the MRI to control the radiation such that when the lesion moves away from its planned position that the radiation will be turned off. There is a group in Edmonton that plans to use a linear accelerator with a magnet to provide real time imaging as radiation treatment occurs. There are patent for using MRI magnets to monitor treatment using electron beams created by a linear accelerator. The problem with this is the non-compatibility of linear accelerators and MRI. Philips has combined with Elektra to develop a prototype MRI/Radiation Therapy system with a linear accelerator in the centre of a magnet and gradient. The magnet and gradient have been elongated to leave a space in the middle so that the linear accelerator unit can be incorporated. The hypothesis is that real time MRI will monitor tumour motion and will guide the radiation therapy to lesion at all times therefore making it motion independent.

U.S. Pat. Nos. 6,198,957 and 6,366,798 (Green) issued to Varian on Mar. 6, 2001 provides a Radiotherapy Machine including Magnetic Resonance imaging system.

U.S. Pat. No. 6,725,078 (Bucholz) issued to St Louis University on Apr. 20, 2004 provides a system combining proton beam irradiation and magnetic resonance imaging

US Patent Application number 20110317812 published Dec. 29, 2011 by David Jaffrey and Mohamad Islam uses two sources of radiation, one to image and the second one for radiation theory.

### SUMMARY OF THE INVENTION

It is one object of the invention to provide a method for guiding radiation therapy which can accommodate movement of the lesion caused by respiration and/or cardiac motion and/or peristaltic motion.

According to one aspect of the invention there is provided a method or apparatus for guiding radiation therapy of a patient comprising:

locating a patient on a treatment support, the patient having a lesion requiring radiation therapy;

preparing the patient for radiation therapy on the treatment support;

while the patient is on the treatment support using an imaging system to obtain a one or more images of a location of the lesion within the patient;

while the patient is on the treatment support using a radiation therapy system to apply a controlled guided dose of radiation to the lesion;

applying to the patient a suitable radioisotope for gamma imaging of radiation emitted by the lesion;

during the application of the radiation therapy obtaining images of the lesion using a gamma camera system responsive to the emitted radiation so as to determine movement of the lesion which occurs during the radiation therapy;

registering the images of the lesion obtained by the gamma camera with said one or more images obtained by the imaging system;

and controlling the dose applied by the radiation therapy system in response to the movement of the lesion detected by the gamma camera system.

The invention also provides an apparatus arranged to carry out and including components so arranged to carry out the above functions

Preferably the imaging system is an MRI system.

However alternatively the imaging system can be a CT system.

Preferably at least one of the gamma images is obtained simultaneously with the imaging by the imaging system.

Preferably the registration of the images is carried out geometrically by physical points on the imaging systems or on the patient support.

Alternatively the registration of the images is carried out by image comparison techniques.

Preferably the control of the radiation therapy is carried out in real time in response to real time images obtained by the gamma imaging system.

Preferably the control of the radiation therapy is carried out by halting the dose whenever the lesion is detected to have moved beyond a predetermined allowable position.

Preferably the control of the radiation therapy is carried out by controlling a focused position of a beam of the radiation therapy in dependence on the movement of the lesion.

Preferably the beam is rotated around an axis and the focused position is moved in a radial direction.

In some cases where the beam is rotated around an axis the focused position is moved in an axial direction.

Preferably the gamma imaging system includes at least two imaging locations spaced around the lesion for generation of a 3-D image of the lesion.

Preferably the radiation therapy is generated by a collimated radiation source which is rotated round the lesion in a manner which controls the application of a required dose of radiation to the lesion while accommodating the shape of the lesion and the movement of the lesion.

Preferably the imaging system is MRI and the method includes the step of moving the magnet of the MRI system away from the treatment support so as to allow the radiation therapy.

Preferably the imaging system is MRI and the method includes the step of moving the patient from the MRI system into the treatment position without patient movement relative to treatment couch.

Preferably the imaging system is CT and the method includes the step of removing the patient from the CT system to the treatment position without patient movement relative to the patient couch.

Preferably the images from the MR system in an MR coordinate system are correlated relative to a coordinate system of the gamma imaging system by using the treatment support as a common baseline.

Preferably the images from the CT system in the CT space are correlated relative to the gamma imaging space by using the treatment support as a common baseline.

Preferably radiation therapy is provided by a radiation source where the radiation source and the treatment support are located in a room shielded to prevent release of the radiation and wherein the room includes a door through which the magnet moves to remove the magnet from the room during the therapy.

Preferably the gamma imaging system is directionally shielded and collimated such that the system is not responsive to scattering of radiation generated by the radiation therapy.

Preferably the gamma imaging system comprises at least one gamma camera heads comprising a collimator, a scintillator, a detector and a read-out system.

Preferably the magnet is an annular magnet surrounding a longitudinal axis and is moved longitudinally of its axis.

Preferably the gamma imaging system comprises a box which shields the radioactive marker.

Preferably the gamma imaging system comprises two cameras at sufficient angles to one another to provide 3 Dimensional distance information.

Preferably the gamma imaging system comprises lead sheets which can be positioned around the cameras and the marker to exclude scattered radiation.

In one arrangement, the gamma cameras operate in constant mode.

In one arrangement, the gamma cameras operate in pulsed mode and there is coordination between the radiation beam and the detection beam such that only one will be in operation at any time.

Preferably there is provided a marker on the patient and one or two additional cameras which monitor the movement of this marker to assist in controlling the guidance of the beam in response to the movement of the body of the patient.

In another arrangement, there is provided a marker on the patient and one or two additional cameras which monitor the movement of this marker and the respiratory marker is used in a training which correlates the position of the lesion with the position of the marker so that the position only of the latter is detected in the RT and used to guide the RT beam.

In another arrangement there is provided an optical marker on the patient and an optical camera which monitors the movement of this marker and the respiratory marker is used in a training which correlates the position of the lesion as detected by the gamma camera system with the position of the marker so that the position only of the lesion is detected and used to guide the RT beam.

Preferably the sets of images of the gamma imaging and the MR imaging are fused together such that the observed gamma image demonstrates all the features of the MRI image.

Preferably the sets of images of the gamma imaging and the CT imaging are fused together such that the gamma images reflect all the features of the CT image.

Preferably images in both modalities are obtained using breath holding or respiratory gating so that the effect of motion is also detected using both modalities and the gamma image is corrected by correcting the MRI image and then fusing these results into the gamma image.

Preferably fused images based on gamma imaging in the treatment position are acquired and fused to provide real time images to detect lesions at all times during the radiation treatment.

### BRIEF DESCRIPTION OF THE DRAWINGS

One embodiment of the invention will now be described in conjunction with the accompanying drawings in which:

FIG. 1 is an isometric view of a patient in an MRI scanner with two multi-head gamma cameras located for imaging radioactive compound concentrated in a tumour.

FIG. 2 is an isometric view of a radiation therapy unit, the location of two multi head gamma cameras to image the radioactive compound concentrated in a neck tumour.

FIG. 3 is an enlarged view of the therapy system and showing one multi-head gamma camera system (7 single head cameras) showing all camera heads directed at a lesion.

FIG. 4 is side elevation view which shows two multi-head gamma cameras.

FIGS. 5A and 5B show the architecture of the basic gamma camera system.

FIG. 6 shows a multi-head architecture diagram.

FIG. 7 shows the multi-head simplified diagram in a flex position with two lesions.

FIG. 8 shows a multi-head detail in flat position including packaging.

FIG. 9 shows a multi-head detail in flex position with an air balloon as the flexing method and packaging.

FIG. 10 shows a detail of a single gamma camera head showing collimator, scintillator, detector and readout board.

FIG. 11 shows an example of a more advanced design of the single camera head.

FIG. 12 shows a simplified diagram of a multi-head design.

FIG. 13 shows the interconnection of the three basic subsystems in this design, which are the position detection system, the image processing system and the position control system.

In the drawings like characters of reference indicate corresponding parts in the different figures.

### DETAILED DESCRIPTION

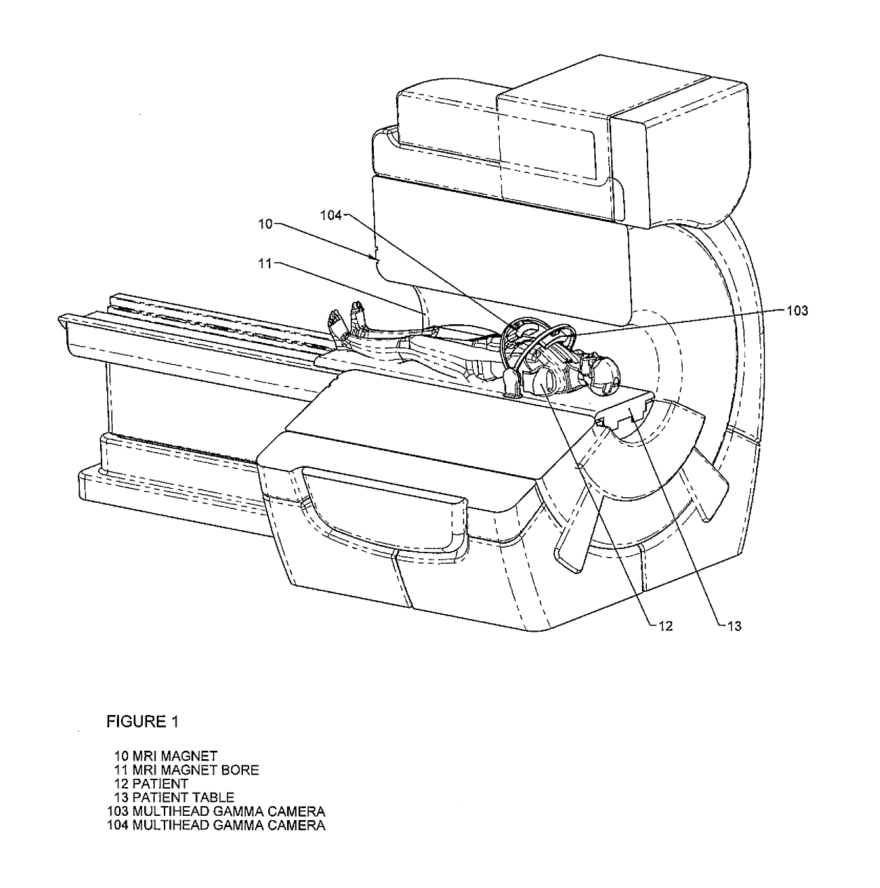

In FIG. 1 is shown schematically a magnetic resonance imaging system which includes a magnet 10 having a bore 11 into which a patient 12 can be received on a patient table 13. This patient table is in fact a component of the patient support system and this is moved with the patient in the identical position on the patient table as the patient moves from imaging device to treatment device. The system further includes an RF transmit body coil which generates a RF field within the bore. The movable magnet is carried on a rail system with a support suspended on the rail system.

The system further includes a receive coil system which is located at the isocenter within the bore and receives signals generated from the human body in conventional manner. A RF control system acts to control the transmit body coil and to receive the signals from the receive coil. The two multi-head gamma cameras, 103 and 104, are held in position using camera holders 116 and 117. The same arrangement would be employed if a patient was imaged outside the treatment room and again, the patient's position relative to the patient support system must remain constant throughout.

As shown, the gamma camera system can enter the magnet and can have MRI visible fiducials such that the gamma camera position is registered to the anatomical images of the patient acquired by the MRI system.

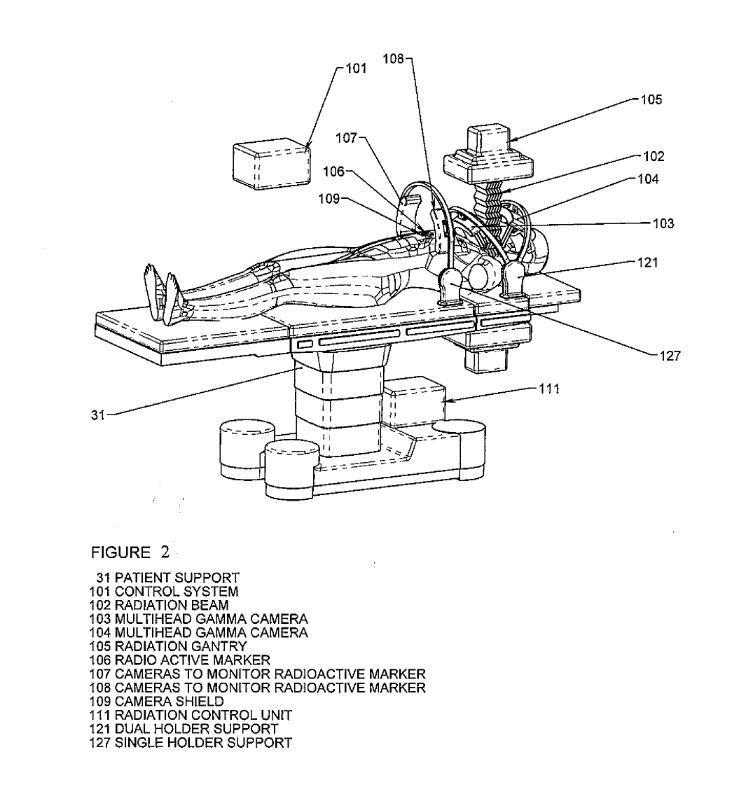

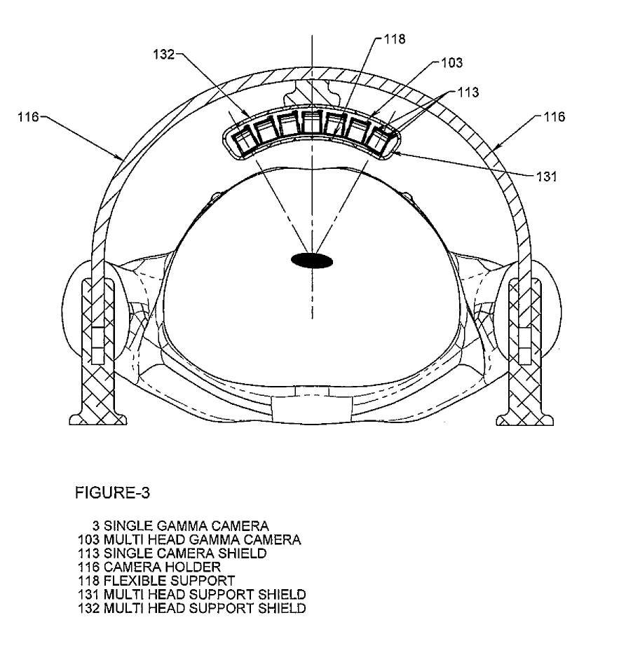

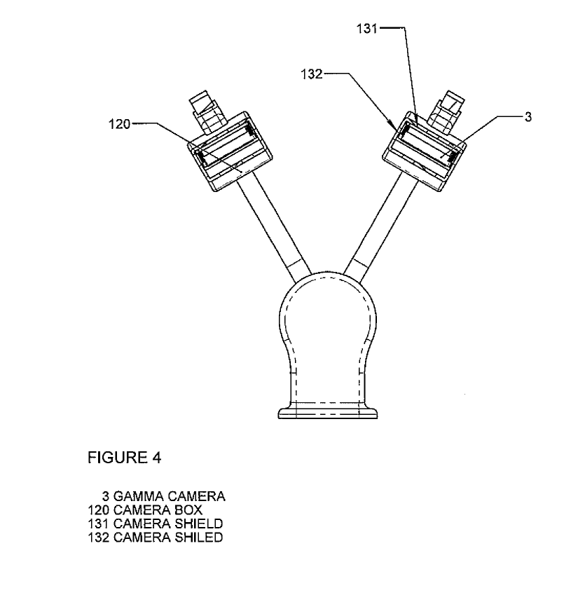

The MRI system is used in conjunction with a patient radiation therapy system shown better in FIG. 2 with the magnet 10 of the MRI system removed or the patient moved from the MRI system to the radiation therapy system on the same couch top. FIGS. 3 and 4 provide perpendicular and expanded views of the patient illustrating the location, support and shielding of the gamma cameras. The therapy system includes a bunker or room within which is mounted a patient support 31 and a radiation gantry 105. The gantry carries a radiation source, which is in most cases a linear accelerator associated with a collimator for generating a beam 102 of radiation. Systems are available for example from Varian where the radiation system and the patient support are controlled to focus the beam onto any lesion of any shape within the patient body, bearing in mind complex shapes of lesion which are required to be radiated.

The patient having a lesion requiring radiation therapy is placed on the treatment support 31 and prepared for the radiation therapy on the treatment support.

During the initial imaging phase, the magnet of the MRI system is carried into the imaging position at the treatment support for imaging the patient while on the treatment support. The magnet of the MRI system is then moved away from the treatment support through a door of the bunker on the rails so as to allow the radiation therapy to commence. Thus the patient is placed on the support or couch which can move such that the electron beam always irradiates the target volume. The gantry rotates such that the focus of the beam is always a relatively small volume. The table can move in three directions and this combined with the rotation focuses the treatment within a specified volume which is arranged o be as close as possible to the margins of the lesion in the patient. The goal is that this volume is the target lesion and only the target lesion. It is required that the entire target lesion receives the same maximum dose of radiation so that all cells within the targeted volume die. It is required that damage to adjacent normal tissue be minimal.

The radiation control unit 111 includes an electrical interface which allows control over its radiation beam over location and time. There is provided a boom system to allow both the radiation unit to be moved sufficiently far from the magnet and moved into position for the radiation therapy.

A system is provided to generate a correlation between the coordinates systems of the patient that is the patient support table, the MR images, the gamma images and the RT beam 101. The latter can be decomposed into the physical location of the radiation therapy unit relative to the patient support table, and the beam coordinate system relative to the radiation therapy unit.

The patient support table is MR compatible, and compatible with the magnet to allow imaging of the region between the head and lower abdomen.

A suitable camera system for the gamma imaging to provide SPECT images is described in the above referenced applications the disclosure of which is incorporated herein by reference so that no detailed disclosure is provided here.

For advanced SPECT imaging using silicon photomultipliers, there is an interest in using small gamma camera systems that can easily be positioned in areas such as the liver, lungs, prostate, the neck, or inside the body for image guided therapy (surgical or radio-). One of the embodiments is the use of gamma cameras to monitor motion during the procedure particularly when radiation therapy is employed.

For this reason, there exists a need to have a simple manner to control the positions of very small gamma camera heads. One method would be to use articulated arms that hold the gamma cameras, however as the number of gamma camera heads increases in a confined space, the logistics of many articulated arms in a small space begin to be excessive. In addition, for a more flexible gamma camera system, the curvature of the SPECT camera would vary depending on the size of the neck, liver, lung, prostate, or other body part. In addition, the curvature of the SPECT system might also vary depending on the size and location of the lesion in question. In addition, the curvature and positioning of the SPECT camera might change depending on the number of lesions in the liver, lung, neck, prostate or other body part, and on the size and spatial distribution of these more than one lesions. In the case of radiation therapy, the cameras must be placed so that 3 dimensional images of the lesion can be detected at all times during the therapy with the gamma cameras outside the pathway of the radiation. For all of these reasons, a more advanced way of positioning small gamma camera heads for improved imaging is desired.

For the neck imaging application case in which the head and neck are immobilized and MRI or CT imaging has already occurred, the location of the suspicious lesion or lesions is already known. MRI is highly sensitive, with sensitivities approaching 100%, and so additional lesions will not be uncovered by the gamma imaging system. Instead, it may be possible to use the gamma imaging system to add additional and improved and differentiated information about the location of the suspicious lesion during the radiation therapy or during surgery so that better intra-procedural decision making can be done.

As an example of a method to optimize lesion imaging during radiation therapy, if the neck lesion is of size 5 mm, and if it is located approximately 3 cm below the surface, one could use a small compact gamma camera of size 50 mm×50 mm to image the volume around the lesion. In this case, if a parallel hole collimator is used, the central portion of the gamma camera will be imaging the lesion volume and the external portion of the gamma camera will be imaging neck tissue where no lesion is found. However, the neck lesion position will change due to motion particularly that a consequence of swallowing. It is necessary to image a portion of the surrounding tissue around the lesion in order to obtain a background count level and in order to obtain an accurate idea of the tumor extent and boundary. The size of the imaging volume would need to be sufficient such that the lesion is imaged at all time and that there is sufficient margin to be able to accurately determine lesion boundaries. The volume of interest would be determined using MRI or CT imaging prior to the procedure. The 50 mm×50 mm cameras could be located to provide a 3 dimensional image of the lesion of this size at all times during the procedure. The gamma images could be used to control the radiation therapy unit so that the focus of the radiation follows the position of the lesion at all times even during the motion or alternatively it could stop the radiation during motion such as swallowing. Of course, if the lesion is of larger size, say 30 mm in a liver, then the entire 50 mm×50 mm gamma cameras face may be pointed in a useful way to image that lesion but not its surrounding tissues particularly during motion. Of course, if a larger gamma camera was used, say 150×150 mm face size, then the lesion and surrounding tissue could be imaged at all times to accurately detect lesion boundaries even during motion. If such a large camera was used for the imaging of the single lesion of size 5 mm which is 3 cm below the surface of the skin then the majority of the gamma camera would not be useful.

One of the fundamental problems is therefore that there may be one or more lesions requiring imaging, and they will be of varying sizes and in various locations in the body organ. In addition, the organ size for different patients will vary from patient to patient and will be at different distance from the body surface. One design approach for the general case is to place multiple gamma cameras to view multiple lesions, however it would be best if these multiple gamma cameras could all view the same lesion in the case where only one lesion needs to be studied, or could view multiple lesions in the case where multiple lesions need to be studied. The cameras would need to provide 3 dimensional images of the lesion at all times and so even for a single lesion multiple cameras may need to be employed. The cameras must always be placed outside the radiation beam used for therapy. This would imply that the cameras would need to be moveable. To do this, one could place multiple cameras on multiple articulated arms. For small lesions of, one might want multiple gamma cameras of size 6 mm×6 mm, arranged in an arc or half-sphere around the lesion being studied. As the size of the lesion decreases and as the distance from the camera position to the lesion decreases, the size of the gamma camera required can also decrease, thereby saving cost. Cabling and wiring, however, may become more complex as the number of gamma cameras increase.

For all these reasons, a more elegant and useful approach to having a flexible positioning system for multiple gamma cameras is to attach the front collimator surface or front package surface directly in front of the collimator for each gamma camera head to a flexible surface that can be flexed to image different lesion geometries, and potentially different sizes of body organs.

A flexibly-mounted multi-headed gamma camera for use in imaging radioactive source distributions is herein described. A large number of small gamma camera heads, with each head consisting of at least collimator, scintillator, detector and read-out electronics or read-out system, are attached to a flexible substrate, and position control and position detection systems are also attached to the substrate and camera systems. The detector is for example a silicon photomultiplier, the scintillator is for example CsI(TI), and the collimator is for example made of lead or tungsten with parallel hole collimation.

In preferred embodiments, the flexible gamma camera has wiring to allow powering, signal readout, clocking and various other functions to occur.

As discussed herein, the flexible gamma camera system can be overlaid on a volume or area of interest, such as, in human imaging, close to the, neck, buttocks, liver, lung, or other portion of the body that is contoured, and this flexible gamma camera will allow a SPECT imaging session to be performed.

The flexible gamma camera system may be located inside a housing which is held away from the source distribution.

In preferred embodiments, as discussed below, the exact positioning of the gamma camera heads is controlled by a position control system, which allows the flexible gamma camera to optimize its imaging for specific functions and needs. The exact position of the gamma camera heads may be determined using any of a variety of means known in the art, including but by no means limited to optical and electromagnetic fiducial methods that are known in the art, as used by Northern Digital, Ascension Technologies, Roper Ind., or other known position measurement systems. Obviously, the gamma cameras are positioned outside the magnet and the radiation therapy unit.

This design architecture is also useful because it moves the processing of the events to be implemented in software, which allows easy software upgrade to the image and signal processing algorithms.

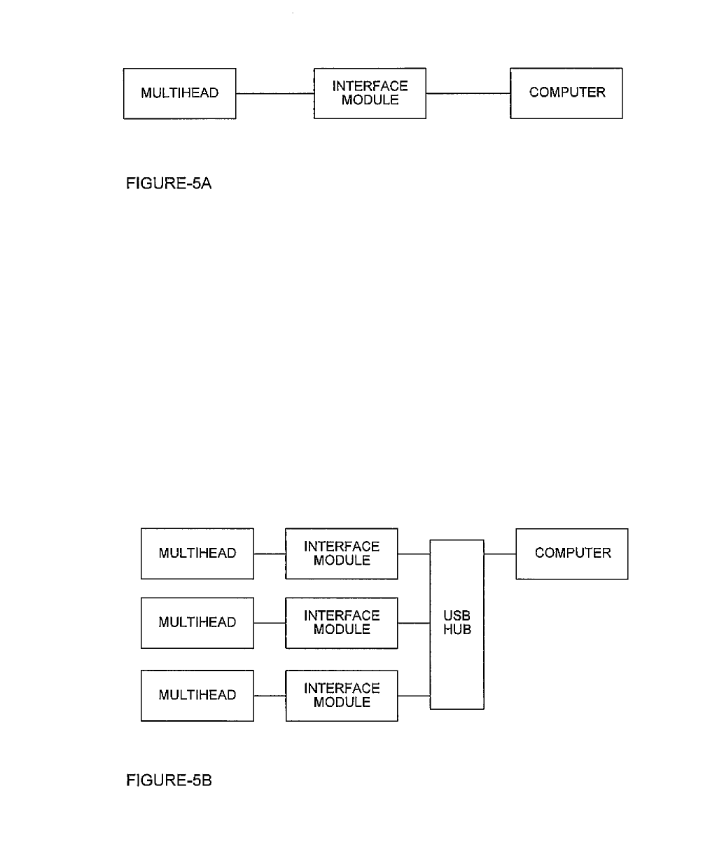

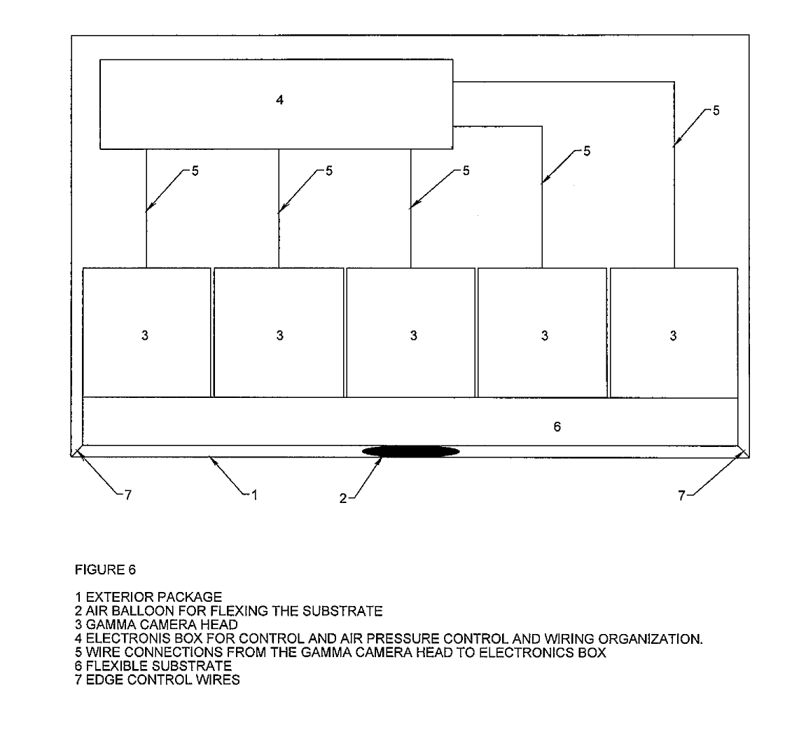

The system is shown in simplest form in FIG. 5a, in which a single multi-head gamma camera is connected to an interface module which is connected to a computer.

FIG. 5b shows an embodiment where more than one multi-head is desired, and in this case there are three multi-heads, three interface modules (IFM), and a USB hub is added to allow networking back to the computer. If the bandwidth requirement of the system is high, Gig E or some other higher speed point to point networking method can be used to connect to the computer. If the processing requirements are high, a higher speed computer may be used to allow the algorithms to perform at the required speed.

FIG. 6 shows a side view of a multi-head. There are five gamma camera heads 3 which connect back to the electronics box 4. The electronics box 4 aggregates the connections for optimum packaging and routing. The electronics box 4 connects to the IFM. The electronics box may also provide the air pressure control, and may provide other control functions such as temperature monitoring, self-test and gamma camera head position measurement. In this configuration the method of camera positioning is to use a balloon which is controlled by an air pump. The air balloon 2 can be inflated and deflated, which moves the flexible substrate 6 up and down, providing more or less curvature. The clearance to the edges of the package determines the maximum amount of curvature that can be used in this design. The edge control wires 7 are springs and pull the edge back tight so that the flexible substrate is returned to a flat configuration from a concave configuration when the air is sucked out of the balloon. These pieces are all held within an exterior package 1. With the use of these edge control wires 7, the air balloon 2 does not need to be glued or attached to the flexible substrate, which allows the air balloon 2 change-out or replacement to be done as easily as possible.

As will become apparent to one of skill in the art, the purpose of this flexibility approach to gamma camera positioning is to allow optimum SPECT imaging to occur in various lesion sizes, orientations, and in various depths and distances from the gamma camera.

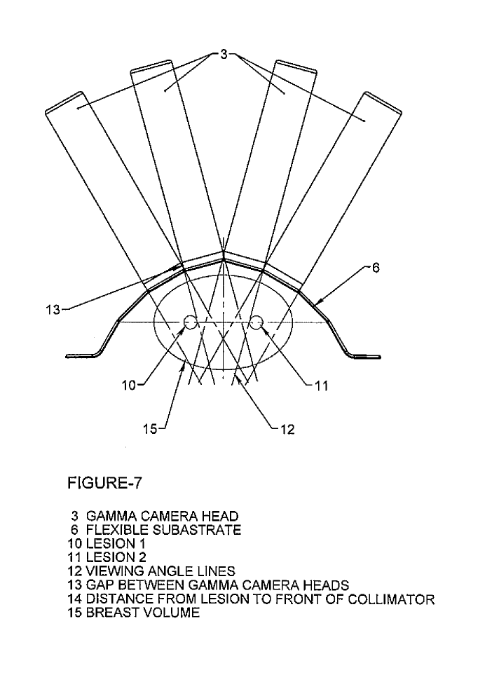

FIG. 7 shows a flexible multi-head being used to image the liver volume 15 which contains two lesions 10 and 11. This level of flexibility in positioning is obtained by using a flexible substrate 6, onto which the multiple gamma camera heads 3 are attached. With this flexing of the multi-head, the entire liver is more optimally imaged, and the two lesions are both imaged by one or more of the gamma camera heads. SPECT image processing can now be used on some of the volume of the breast. The gap between the gamma camera heads 13 remains the same distance, because the gamma camera heads are firmly affixed to the flexible substrate. The flexible substrate does not flex throughout its distance, because the front of the gamma camera heads use a rigid lead collimator, and so the flexible substrate can only bend at the locations between the gamma camera heads.

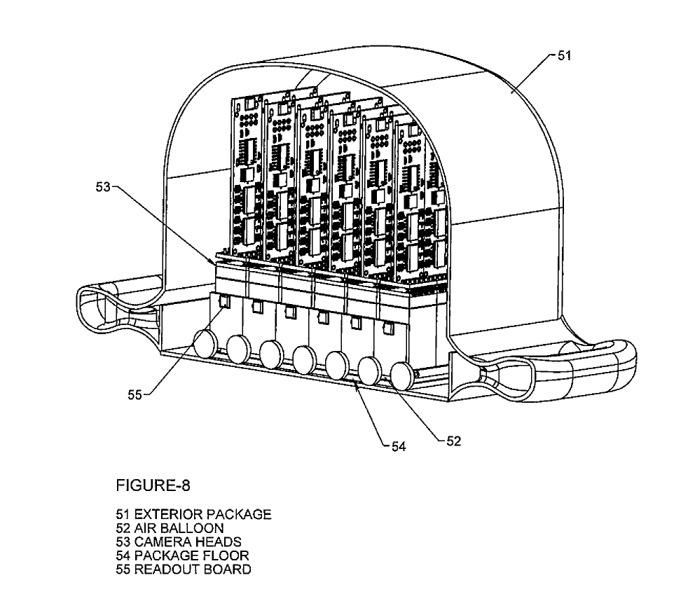

FIG. 2 is the rendering from the diagram in FIG. 8 from a isometric perspective. One of the 4 cameras shown in FIG. 2 is shown illustrating the angle that it makes with the body surface so that the camera is outside the beam 102 from the high energy radiation source. A second multi-head camera 104 could be placed on the other side of the radiation beam as shown in FIG. 1. The angles that the multi heads make with the body surface would be optimized to minimize scattering from high energy radiation and maximize the measurement of lesion position. The camera heads would be able to monitor any motion in any direction of the lesions and this would be transmitted to the controls of the radiation device

FIG. 8 is a rendering of a multi-head that allows curvature in 2 dimensions to occur. The exterior package 51 is used to hold multiple gamma camera heads 53. The air balloon 52 is located against the bottom of the package. The bottom of the package 54 does not curve, but is instead maintained flat and is sufficiently rigid to provide mechanical stability as the air balloon provides curvature to the gamma camera multi-head system. Extending off of the back of each camera head is the readout board 55 appropriate for that camera head. The readout boards are connected to the electronics system, which then connects to the interface module IFM. In this embodiment, the air balloon is connected via an air tube to a simple pump ball mechanism, which allows the user to adjust the bend of the gamma camera. The readout wires, fiducial wires, air tubing, and electronics box are not shown in this rendering.

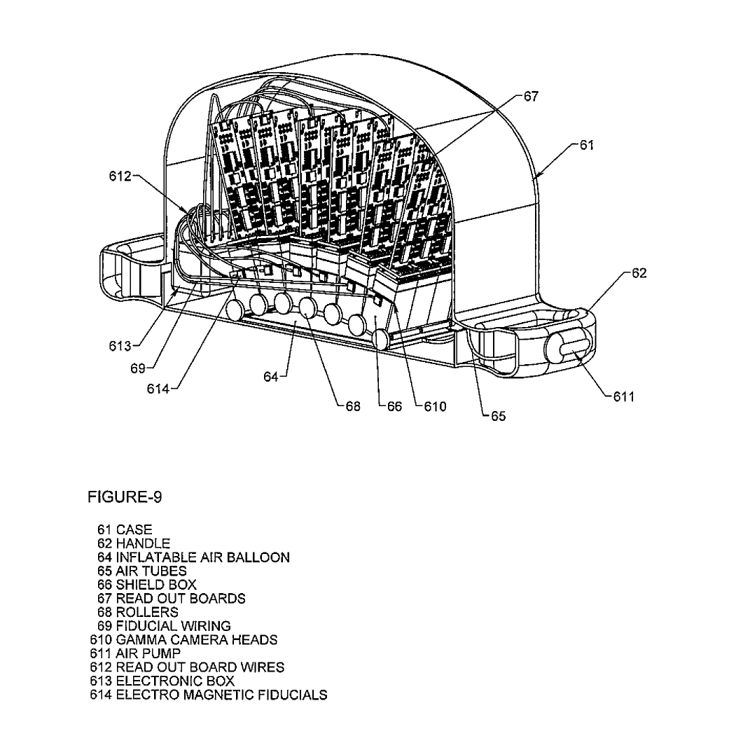

FIG. 9 shows the same multi-head in additional detail with the flex operation occurring. In this case, the inflatable air balloon 64 is inflated using air pump 611 via air tubes 65, with the air pump located on the handle 62. Each row of gamma camera heads 610 is located within a lead shield box 66. The shield box may alternatively be made from tungsten. The row ends of the gamma camera heads roll on rollers 68 to allow easy movement of the ends, because the ends are pulled into towards the middle of the gamma camera as the flex occurs. The gamma camera heads comprise collimator, scintillator and detector 610 and readout boards 67. The shape and size of the exterior case 61 determines the allowable amount of flex, because the readout boards 67 cannot hit the side of the case. The amount of flex is determined by the stops that are designed within the enclosure. These stops are not shown. The amount of flex that occurs can be determined using electromagnetic fiducials 614 and fiducial wiring 69. The EM fiducials operate within the measurement volume dictated by the magnet system, not shown, as is normal for Ascension Technologies and other electromagnetic position measurement systems. The amount of flex determines the amount of overlap that exists between the collimator holes, which therefore determines the amount of imaging improvement that will occur in this design. The electronics box 613 aggregates and coordinates the read out board wires 612, the fiducial wires 69, and provides command and control and interconnection functionality back to the IFM box. The IFM box and the connection wiring to the multi-head are not shown. The air pressure release valve is also not shown.

As will be apparent to one of skill in the art, there are several algorithmic approaches that can be used in determining how much flex to provide to the surface and are well within the knowledge of one of skill in the art. For example, one simple approach is to monitor the sensitivity of the gamma camera with the surface flat, and then to flex a little bit, and watch the sensitivity to see if it improves. This approach can be incorporated into an automatic control system, and so the user can turn on the flex button and the system will self-flex.

For lesions that are smaller than the total size of the gamma camera, flexing will always lead to an improved sensitivity. In the example where lesion movement is occurring, as is the case for radiation treatment, the imaging volume must include the lesion in all of its positions. For example, if the multi-head has 4 gamma camera heads of 20 mm square each, making the entire multi-head a linear distance of 80 mm, then any lesion in all positions smaller than 80 mm can benefit from having some flex in the system. In the case where the lesion is 70 mm in size, you would expect only a little flexing to provide the largest sensitivity, whereas in the case where the lesion is quite small, say 10 mm in size, the system can flex a lot before the highest sensitivity is achieved. As will be appreciated by one of skill in the art, any suitable algorithm will have stops built into the software so that the system is not over-flexed. There will also be mechanical stops built into the system to ensure that the flex does not go too far, causing the PCB boards to hit the outside of the case, or causing the flexible material to be over-flexed, as discussed above.

For any position of the multi-head, the magnetic position measurement system can automatically measure the position of all of the elements, using methods known in the art, for example, as available from Ascension Technologies or other similar companies.

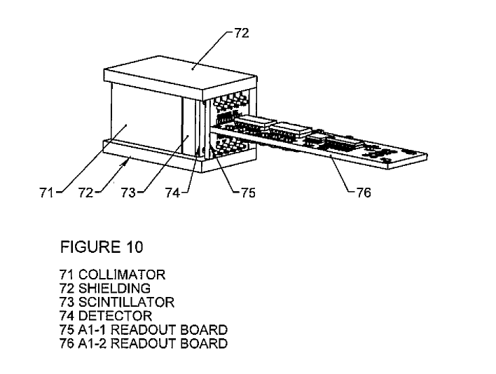

FIG. 10 shows a typical camera head, which shows one version of the CSDE (Collimator, Scintillator, Detector, read out Electronics) arrangement. The collimator 71 is 23 mm deep, the scintillator 73 has a thickness of 5 mm, the detector 74 is about 2 mm in depth with pins off the back, a small PCB 75 A11 readout board is used in the back of the detector to organize the connections, and the larger PCB 76 A12 readout board is inserted into the connection organization PCB to allow the read-out electronics to be used. A common scintillator that can be used in this case is CsI(TI) pixelated on approximately 3.5 mm pixellations, in order to match up with the pixel size of the Array 4 detector from SensI. On the four sides are located lead shielding 72 of thickness 1.5 mm.

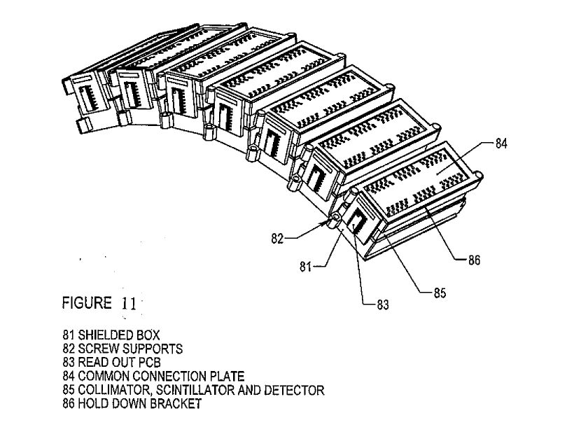

Further advances can be made with packaging as one reduces the size of the PCB read out boards via integration. FIG. 11 shows a flexible array in which the PCBs have been shrunk in size and repositioned along the side of the row. The diagram shown in FIG. 3 is an example of the application of this type of configuration to image the liver of a patient. There are 7 rows of 3 Array 4's each, however over the top of the row is a common connection plate 84 that connects all of the pins on the back of the Array 4 into the read out PCB 83 that is on the side of the row. As well, in this case a different collimator depth of 10 mm is used, instead of the 23 mm previously used in the other Figures. As the collimator depth decreases, the noise level that penetrates the collimator increases, and so noise reduction and anti-noise software needs to be employed to ensure good performance. The multi-head array in FIG. 10 is based on the Sensl Array 4 which is nominally 15×15 mm square and 2 mm high. The Array 4 is available in various package styles, including pins grid array and ball grid array which allows a lower profile. The common connection plate 84 over the top of 3 detectors provides additional mechanical stability as well as electrical connection, and the common connection plate connects to the readout PCB on the side of the row. This design also uses lead shielding boxes 81, screw supports 82 that allow the rows to be assembled, and a hold down bracket 86 through which the screw support is used. The collimator, scintillator and detector 85 are just visible through the gap between the lead shielding box and the hold-down bracket. This arrangement and assembly can be flexed by hand by the operator, and will typically have an external package over top that allows the flexibility to occur. The wiring is not shown. The electromagnetic or optical fiducials in this case are also not shown. This multi-head will typically be used, for example, with a neck surgical patient in which the magnet for em position measurement is located below the supine patient's neck area, the patient then lies down on the table, and then the flexible multi-head is placed on the patient's neck area to be imaged the multi-head is connected to the interface module IFM.

The system described includes three main interacting subsystems: the gamma imaging system, the position detection system, and the position control system. The gamma imaging system consists of multiple gamma camera heads that are held in some position, the IFM digital processing system, and the computer processing system. The position detection system consists of fiducials attached to the gamma camera heads, a magnet located below or above or in the vicinity of the item being imaged, and the hardware and software required for the EM measurement system in the way that is standard in the art, as is provided commercially by Ascension Technologies. The position control system in this a simple case is an air bag that can be filled using a ball pump, and which can allow the air to be released using a release valve. The position control system may or may not include software algorithms to automatically provide movement of the camera, or to provide suggestions and feedback to the operator, or the position control system can simply allow the user to pump the air balloon herself. Other methods to position the system can be envisaged but it is essential that once positioned the system remains solid during the radiation therapy since its position will have been detected and marked prior to the commencement of treatment.

The image processing system uses position information from the position detection system to allow it to do SPECT processing or other processing, but does not need any information from the position control system. If additional curvature is required, additional air can be pumped into the air balloon. The image processing system may provide guidance to the operator in order to indicate whether more or less curvature, and air, is required within the bladder. Alternatively, the only guidance provided may be the sensitivity measurement as discussed above.

The position control system may have a priori knowledge of the location of lesions from CT or MRI anatomical imaging sessions, or there may be no a priori information.

The lesion being imaged may be selected from the group consisting of a neck lesion, a sentinel lymph node, a lymph node in the lymphatic system, or some other lesion of interest. Any lesion or body part of interest that can benefit from a system that maximizes the sensitivity of the captured radiation emissions is suitable for this type of system.

One aspect of this invention is that the amount of flex curvature required is different for different situations and for different patients, and so the gamma camera system described can be used to suit a wide variety of patient needs.

Another reason for providing the flexibility for this system is that altering the viewing angle for the volume in question can sometimes lead to changes in the noise levels that are received, which can also allow an improvement in imaging to occur. During radiation therapy procedure, the patient may be imaged prior to the introduction into the treatment room and this can be used to optimize imaging characteristics

Another reason for providing this flexing is in the case of the “second” lesion 11 as discussed above, in which a lesion is located at a midpoint in the gap between imaging elements. In this case, the flexing of the surface allows different viewing angles to be used for the same volume, leading to improved and more complete imaging of the volume in question. As discussed above, when the surface is flexed, the positions of the camera heads can be measured using an electromagnetic method that allows complete knowledge of the camera head position to be achieved, and which therefore allows SPECT image processing to occur in which multiple cameras view the same volume, leading to 3d SPECT images.

It is also important to note that the input of air to the air balloon can be placed under computer control, and the position control system may therefore use automated algorithms and methods to provide optimized imaging.

The multi-head will typically require shielding of the scintillators and detector areas, and this can be accomplished either by encasing all of the multi-head in an enclosure, and the enclosure has shielding, or each individual camera head can have lead shielding. This approach can be used in the case where one wants the maximum flexibility of positioning.

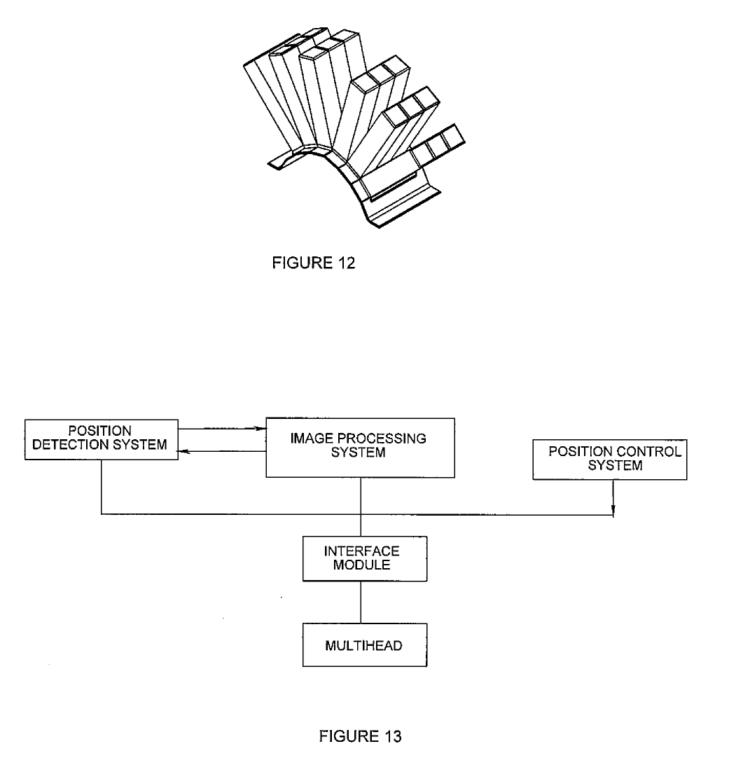

FIG. 12 shows a multi-head without the PCBs showing, but instead has an enclosure around each camera head. In this case, each small enclosure has lead shielding, and this system requires more space between the camera heads than a system in which has already been described. Typically, there will be 1.5 mm of lead shielding on all four sides, and so if the package thickness is 1 mm and the lead is 1.5, this leads to a total distance between the camera heads of 5 mm, which might allow for “second” lesion type situations to arise.

For each of these designs, the lead shield box of the collimator is typically glued or attached onto the flexible surface. This approach to a flexible gamma camera with closely spaced gamma camera heads is only possible for concave movements, because if the surface goes convex then the readout boards will hit each other. If convexity is desired, the gamma camera heads can be spaced further apart to allow this movement to occur. The amount of convexity will be related to the spacing between the camera heads and the height of the readout portion of the camera heads. The height of the camera heads can be modified by moving the PCB to the side as presented in this discussion. The lead shields for use in the radiation treatment room can slide along grooved placed at the appropriate positions along the edges of the boxes which hold the detector etc. This additional shielding is in place to minimize any scattering from the high energy radiation source from entering the camera and disturbing the scintillator. The camera holders are modified to allow this additional shielding.

The positioning measurement system has been discussed in magnetic terms as is common with Ascension Technology methods, and this means that a magnet must be somewhere close to the multi-head so that the position of the heads can be measured. The magnet needs to be removed for patient MR imaging and then returned to the identical position. In the case of the hand positioned camera blanket, if optical fiducials are used on the camera heads then optical position measurement is possible, in any of a variety of manners known in the art, for example, as long as there is the appropriate infrared camera system as is available from Northern Digital. Optical cameras cannot enter the MRI magnet and so fiducials which are visible both optically and in MRI must be strategically placed so that the optical image space can always be correlated with the MR image space.

The gamma camera elements are made up of a collimator, scintillator, detector and suitable electronics for analog and digital signal processing, powering, status and mode control and other functions. Using technology available in simple design methods today, in some embodiments, the gamma camera will be 80 mm to 120 mm in height and approximately, for example, 14×14 mm in length and width. These are the typical dimensions of the Cubresa GCH1501 design. In these embodiments, the height of the gamma camera would consist of 20 mm for the collimator (a lead collimator available from Nuclear Fields, for example), 5 mm for the scintillator (a CsI(TI) scintillator that allows approximately 80% stopping power at 140 keV energies, for example), 10 mm for the detector (which might be a SensL Array 4), and the remaining depth used for the electronics boards, cabling and connectorization, and packaging space. This is a high, narrow camera element. By placing many elements on a flexible surface, all next to one another, it is possible to make a flexible camera surface. The camera elements can be flexed easily in various directions so that different lesion sizes and shapes can be optimally imaged.

Having many camera elements on the flexible surface may make it necessary to have an interconnection system on the rear of the elements that provides mechanical support. The common connection plate is one example of a method. This is used so that undue moment of force does not cause damage to the system or cause the elements to fall over. The collimator will be the majority of the weight, and therefore the majority of the weight can be expected to be near the rubbery surface, and so the force moment may not be too high.

As will be readily apparent to one of skill in the art, there are various applications for this camera system.

As discussed herein, a flexible gamma camera system is described which will provide optimal imaging for various lesion requirements. The flexible gamma camera system can be used with an MRI system or by itself. It can use the MRI system to provide it with lesion information on which to curve the surface, or it can first image in the flat orientation and then curve the surface itself based on imaging results. This method can be used with various gamma energies, and therefore is suitable for various applications.

The curved surface will typically exist within a larger package, and the larger package will provide flexing hardware to allow the surface to flex.

The flex surface can be made of various materials and in various ways, including a firm rubber on which all of the gamma camera collimators and elements are glued, and including a metal or plastic hinge system that allow for the interconnection of all the collimators. For the case of the metal hinge system, as long as the metal hinges do not interfere with the front of the collimators, it is possible to use them. Also, metal hinges would not typically be used within a high magnetic field area such as near an MRI, unless the metal is of suitable metal that is not magnetic. It is also possible to embed a rubber grid between the collimator surfaces.

Also, the connection between Array 4 and electronics does not need to require the electronics card to be directly plugged into the Array 4. Instead, a ribbon cable or suitable connectorization and cabling can be used between the Array 4 and the electronics to allow the electronics to be positioned further away from the Array 4, which will allow easier packaging options and movement options in some applications.

Alternatively, instead of using a flex surface on which gamma camera elements are mounted, it is possible to use a flexible surface in which hinges or hinged surfaces between the gamma camera elements are used. In an application where the camera must stay on a plane and cannot be flexed as a whole, the individual elements, CSD (collimator, Scintillator and Detector) may be hinged or affixed onto a membrane in order to focus on the lesion of interest. The CSD will be moved either manually or with the use of a focusing driver/operation. The CSD will be connected to the electronics by a grouping of wires or cables in order to transfer the signals, shown below.

The flexing systems that have been discussed within this invention have illustrated 2-dimensional concave flexing.

The position measurement system can use the known orientation of the bottom of the gamma camera heads to improve the accuracy of the measurement. Unlike an object that can float freely in space, it is known a priori that the gamma camera elements are attached to the flexible substrate, and that the gamma camera elements remain side by side. For this reason, the convergence of the positioning algorithm may be that much quicker because of this known orientation between the gamma camera heads.

These gamma camera heads each have approximately 10 wires coming from the back, with the wires carrying 2 output signals, carrying in 4 voltage and ground signals, and carrying in a reset and clock line. In addition, monitoring and communications may require another 2 lines, and therefore in a typical case the readout boards have 10 wires connections. These 10 connections from each readout board are routed off of the gamma camera flexible substrate to a connection aggregator. The connection box allows a connection to occur to the external digital IFM system via an interface containing 4 power and ground lines, 2 lines for reset and clocking and 2×18=36 signal lines, for a total of 42 lines. The 4 power and ground lines are shared by all 18 gamma camera heads, the 2 reset and clock lines are also shared between all gamma camera heads, and the 2 signal lines from each gamma camera head must be routed back in their totality to the digital processing systems contained within the PC.

FIG. 13 shows the interconnection of the three basic subsystems in this design, which are the position detection system, the image processing system and the position control system. It indicates that the position movement system and the position measurement system are separate systems. In some designs, the position movement system consists of an operator positioning the unit by hand.

As part of the design of the multi-head, MR, X-ray, Gamma or optical fiducials may be used to allow image registration for multimodal image analysis and presentation. For example, in the case where MR and gamma images will both be used with a patient, it is possible that the multi-head may be attached to a prostate biopsy system, such as a square grid with fenestrations, and that the square grid will already have MR fiducials as part of its design. Once the multi-head is attached on the square grid, the multi-head package position would also be known via mechanical registration, and then the flexing portion of the multi-head can be referenced to this known position.

The arrangement as described herein therefore provides camera systems 107 and 108 and software control 101 to add GI to the radiation system such that the position of the tumor in the body can always be known and this information can be transmitted rapidly to the control system 111 directing the radiation beam. The patient is injected with a tumor targeting radioactive substance such as Tc99 Sestimibi or similar radioactive compounds. The choice of radioactive compound is guided by the type of tumor to be irradiated.

The beam 102 as the gantry 105 rotates is cylindrical in shape as shown in FIG. 2. The gamma cameras 103, 104 are located at angles such that their zone of detection other than the actual lesion is outside the range of the cylindrical radiation beam. In one of the embodiments there will be two gamma cameras 103, 104 located at an angle A of 45 to 135 from one another so that movement in all 3 Cartesian directions can be detected and quantified. The camera systems 103 and 104 are held in position by a camera holder, 116. The camera holders are attached to the table by a single holder support, 127 or a dual holder support, 121. These supporters and the holders are radiolucent. And so will transmit the radiation beam as does the patient table. Two of the supports, 127 can be used in place of the double one, 121, and separated by the width of the radiation beam if necessary. The software 101 makes from the images of the two (or more) cameras a pseudo 3 dimensional representation from these two projections.

The camera heads are held in boxes which can have lead sides and lead opposite the lesion direction. These boxes can be made in a curved fashion for the face towards the lesion as seen in FIG. 3 or flat as seen in FIG. 4. In both configurations, it is possible to have a balloon (120) below the camera multi heads to further increase the concave nature of the configuration. This is an example of that shown in FIG. 8. It is possible to employ two balloons so that the camera multi-heads can be concave in both directions as suggested in FIG. 9.

The radiation beam can either be pulsed or constant. This allows the imaging by the cameras to be sequential to the beam (milliseconds to seconds alternation) or simultaneous with the beam. The gamma cameras can operate in either pulsed or constant mode. When in pulsed mode there is coordination between the radiation beam and the detection beam such that only one is in operation at any time.

In one embodiment there is a radioactive marker 106 on the chest of the patient and so that there is provided one or two additional cameras 107, 108 which monitor the movement of this marker. The two additional cameras provide a 3 dimensional representation of the marker movement.

Each of the camera heads includes a lead box 113 or shield which covers the camera(s) apart from an entry end so that the camera is directionally shielded. This acts to attenuate significantly any scatter radiation from the high power radiation therapy. There is a lead shield 109 at the marker between the marker and the body of the patient which shields the marker from the patient and more importantly the patient from the marker. This is shown in FIG. 2.

The radiation frequency of this marker is preferably different by using a different radioactive isotope than that used to measure the location of the lesion. During a training session, the typical location of the lesion relative to the chest marker is determined so that this information can guide the lesion detecting camera(s) during radiation therapy.

In one embodiment of the patent, the use of this respiratory marker 106 to guide the radiation therapy provides unique image guidance in the therapy vault. The training will correlate the position of the lesion with the position of the radioactive marker 106 so that the position only of the latter can be detected in the RT and used to guide the RT beam.

The emission of gamma rays from the patient can be masked by the very high energy radiation of the beam. The high energy beam is directed at the lesion but there will be scatter and some of this scattered radiation can enter the camera unless filters are applied. The filters are software in nature in the control system 101. The attenuation of scattered radiation is minimized by the appropriate use of lead shields 113 of 3 mm in thickness around the camera heads. Lead shield may also be incorporated, 131 and 132, into the boxes 129 which carry the camera multi heads. In one embodiment of these boxes, the sides may be extended so that shielding down to or up to the body surface may be obtained.

The gamma images from the lesion guides the treatment in one of two ways. In the first, the radiation is simply turned off when the lesion moves away from the designated killing zone as detected by the imager. In the second, real time images are transmitted to the radiation device to modify the direction of the beam such that it is always on target. The direction can be controlled by the gantry in a radial direction R to change the radial position of the focus F. Alternatively or in addition, software control of collimators can change the intensity of the beam either radially or axially. to change the axial and radial positions of the focus.

It is known that on average the best tumor delineation within a human body is obtained using MRI. Ideally prior to radiation, the patient has an MRI image followed immediately by a gamma image. The two sets of images are co-registered using mechanical registration methodology. The two sets of images can be acquired simultaneously. The sets of images are fused together such that the observed gamma image demonstrates all the features of the MRI image. High resolution images in both modalities are obtained using breath holding or respiratory gating. The effect of motion is also be detected using both modalities and where useful the gamma image is corrected by correcting the MRI image and then fusing these results into the gamma image.

The invention can provide much better control of radiation treatment of tumors located in regions of the body subject to motion. It can result in a bigger cell kill per grey of irradiation. It can minimize the irradiation of normal tissue adjacent to the tumor lesion and hence collateral damage.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. A method for guiding radiation therapy of a patient comprising:
locating a patient on a patient support device, the patient having a lesion requiring radiation therapy;
preparing the patient for radiation therapy on the patient support device;
while the patient is on the patient support device using an imaging system to obtain a one or more images of a location of the lesion within the patient;
while the patient is on the patient support device using a radiation therapy system to apply a controlled guided dose of radiation to the lesion;
applying to the patient a suitable radioisotope for gamma imaging of radiation emitted by the lesion;
during the application of the radiation therapy obtaining images of the lesion or of a location on the body of the patient correlated with the lesion using a gamma camera system responsive to the emitted radiation so as to determine movement of the lesion which occurs during the radiation therapy;
registering the images of the lesion obtained by the gamma camera with said one or more images obtained by the imaging system;
and controlling the dose applied by the radiation therapy system in response to the movement of the lesion detected by the gamma camera system.

2. The method according to claim 1 wherein at least one of the gamma images is obtained simultaneously with the imaging by the imaging system.

3. The method according to claim 1 wherein the registration of the images is carried out geometrically by physical points on the imaging systems or on the patient support device.

4. The method according to claim 1 wherein the registration of the images is carried out by image comparison techniques.

5. The method according to claim 1 wherein the control of the radiation therapy system is carried out in real time in response to real time images obtained by the gamma imaging system.

6. The method according to claim 1 wherein the control of the radiation therapy system is carried out by halting the dose whenever the lesion is detected to have moved beyond a predetermined allowable position.

7. The method according to claim 1 wherein the control of the radiation therapy system is carried out by controlling a focused position of a beam of the radiation therapy system in dependence on the movement of the lesion, wherein the beam is rotated around an axis and wherein the focused position is moved in a radial or an axial direction.

8. The method according to claim 1 wherein the gamma camera system includes at least two imaging locations spaced around the lesion for generation of a 3-D image of the lesion.

9. The method according to claim 1 wherein the imaging system is MRI and wherein a magnet of the MRI is moved away from the patient support device so as to allow the radiation therapy.

10. The method according to claim 1 including moving the patient from the imaging system to the radiation therapy system without moving the patient position relative to the patient support device.

11. The method according to claim 1 wherein the images from the imaging system in a coordinate system of the imaging system are correlated relative to a coordinate system of the gamma camera system by using the patient support device as a common baseline.

12. The method according to claim 1 wherein the radiation therapy system comprises a radiation source where the radiation source and the patient support device are located in a room shielded to prevent release of the radiation and wherein the room includes a door through which a magnet moves to remove the magnet from the room during the therapy.

13. The method according to claim 1 wherein the gamma camera system is directionally shielded and collimated such that the system is not responsive to scattering of radiation generated by the radiation therapy system.

14. The method according to claim 1 wherein the gamma camera system comprises at least one gamma camera head comprising a collimator, a scintillator, a detector and a read-out system.

15. The method according to claim 14 wherein the gamma camera system comprises a radioactive marker which is mounted in a shielded box which shields the radioactive marker and which can optionally slide around the camera head.

16. The method according to claim 1 wherein the gamma camera system comprises two camera heads at right angles.

17. The method according to claim 1 wherein the gamma camera system operates in constant mode.

18. The method according to claim 1 wherein the gamma camera system operates in pulsed mode and there is coordination between a radiation beam of the radiation therapy system and a detection beam of the gamma camera system such that only one will be in operation at any time.

19. The method according to claim 1 wherein there is provided a marker on the patient and one or two additional gamma camera heads of the gamma camera system which monitor the movement of this marker to assist in controlling the guidance of a beam of the radiation therapy system in response to the movement of the body of the patient.

20. The method according to claim 1 wherein sets of images of the gamma camera system and the imaging system are fused together such that the observed gamma image demonstrates all the features of the image of the imaging system.

21. The method according to claim 1 wherein images in both the gamma camera system and the imaging system are obtained using breath holding or respiratory gating so that the effect of motion is also detected using both the gamma camera system and the imaging system and a gamma image is corrected by correcting an image from the imaging system and then fusing these results into the gamma image.
